(19) United States
(12) Patent Application Publication
(10) Pub. No.: US 2022/0287965 A1
(43) Pub. Date: Sep. 15, 2022

(54) OLEOGELS AND METHODS RELATING THERETO

(71) Applicant: North Carolina Agricultural and Technical State University, Greensboro, NC (US)

(72) Inventors: Salam Ibrahim, Greensboro, NC (US); Tahl Zimmerman, Greensboro, NC (US); Roberta Claro da Silva, Greensboro, NC (US)

(21) Appl. No.: 17/692,288

(22) Filed: Mar. 11, 2022

Related U.S. Application Data
(60) Provisional application No. 63/159,737, filed on Mar. 11, 2021.

(51) Int. Cl. A61K 9/06; A61K 45/06; A61K 47/44; A61K 35/741; A23L 33/135

(52) U.S. Cl. CPC A61K 9/06; A61K 45/06; A61K 47/44; A61K 35/741; A23L 33/135

(57) ABSTRACT

The presently disclosed subject matter relates generally to oleogels, particularly to oleogels that can be used for delivering active ingredients such as probiotics. Also described herein are methods for preparing and using such delivery systems.

### CROSS REFERENCE TO RELATED APPLICATIONS

This application claims priority pursuant to 35 U.S.C. § 119(e) to U.S. Provisional Patent Application Ser. No. 63/159,737 filed Mar. 11, 2021, which is hereby incorporated by reference in its entirety.

### STATEMENT AS TO RIGHTS TO INVENTIONS MADE UNDER FEDERALLY SPONSORED RESEARCH OR DEVELOPMENT

This invention was made with Government support under NI191445XXXXG007 awarded by the U.S. Department of Agriculture. The Government has certain rights in this invention.

### TECHNICAL FIELD

The presently disclosed subject matter relates generally to delivery systems such as oleogels that can be used for delivering active ingredients such as a probiotic, microbial cell, enzyme, vitamin, micronutrient, antimicrobial agent, and any combination thereof. The presently disclosed subject matter also relates generally to methods for preparing and using such delivery systems.

### BACKGROUND

One issue facing the probiotics industry today is how to reliably deliver high quality volumes of viable probiotic bacteria, which have been identified as delivering health benefits to the consumer. Maintaining viable bacterial cells is a key characteristic of a functioning probiotic delivery system because consumption of at least 7 log viable cells (i.e., 7 Log Colony Forming Units per milliliter or 7 Log CFU/mL) has been suggested to provide health benefits to consumers. Another challenge to the delivery of an effective amount of probiotics is survival of the probiotics through the digestive tract, and particularly protection from destructive gastric juices, so that the probiotics are delivered to the gut where they have been shown to have positive effects.

Current probiotic product delivery systems have limited value, as they typically show loss of cell viability and functionality over time during storage, even with refrigeration. This loss of viability and functionality is observed with both supplement-based delivery systems, which generally rely on freeze drying, and food-based delivery systems, in which probiotics are added directly to food, such as dairy products, which typically use spray drying techniques. Freeze-drying is expensive, energy intensive, and most importantly, has not been shown to provide long-term storage efficacy. In one example, the dairy industry used spray drying to prepare starter cultures in the production of yogurt and cheeses, and tried to incorporate probiotic supplements into the production; the viability of the probiotics was low and the formulation unstable over time. When the dairy product is yogurt, there is competition between added probiotic strains, as well as challenges requiring modifications to the fermentation process. Loss of cell viability in the production and storage steps leads to a decrease in health benefits to the consumer.

Similar challenges have been identified in the delivery of microbial cells (e.g., cells not classified as probiotics), enzymes (such as lactase or glucosidase), vitamins, micronutrients, antimicrobial agents, and combinations thereof.

Encapsulation is the process of trapping functional agents in a carrier material, such that the carrier effectively controls and directs release of the functional agent. Encapsulation of a functional agent (e.g., an active ingredient) can effectively isolate the functional agent from the surrounding environment. Encapsulated functional agents can more readily be incorporated into food and can survive exposure to gastric juices, thereby maintaining their viability.

While current technologies may have measurable probiotic carrying capacity (the ability to encapsulate bacteria), in general probiotic recoverability (the proportion of recoverable viable cells) from those formulations is too low over the long term to provide health benefits to the consumer. Thus, the functionality of probiotic-containing products is limited. Currently known encapsulation techniques for incorporating probiotic strains into food products are limited in utility because they do not promote stability, that is, they cannot maintain the probiotic recoverability and functionality of the probiotic strains over extended storage periods. In addition, current encapsulation methods do not demonstrate good resistance to degradation due to gastric juices in vivo.

Accordingly, there is still a need for a universal, cost-effective and efficient encapsulation system for delivering active ingredients such as those that can be incorporated into food products. Such a technology would also have an application for the storage and delivery of active ingredients.

### SUMMARY

A first aspect of the present invention is directed to an oleogel comprising an oil, a wax, and an oleogelator.

Another aspect of the present invention is directed to an oleogel emulsion.

A further aspect of the present invention is directed to a composition comprising: an active ingredient; and an oleogel comprising an oil, a wax, and an oleogelator, wherein the active ingredient is within the oleogel. In some embodiments, the active ingredient is within an aqueous phase present in the composition, optionally wherein the aqueous phase is dispersed within an oil phase of the oleogel.

An additional aspect of the present invention is directed a method of preparing a composition comprising an active ingredient, the method comprising: forming an oleogel comprising a wax, an oil, and an oleogelator; and combining an active ingredient and the oleogel to form the composition comprising the active ingredient.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIGS. 1A-1B are illustrations depicting an example oleogel preparation according to some embodiments of the present invention.

FIG. 2 is an illustration depicting an example oleogel formulation according to some embodiments of the present invention.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All references listed herein, including but not limited to all patents, patent applications and publications thereof, and scientific journal articles, are incorporated herein by reference in their entireties to the extent that they supplement, explain, provide a background for, or teach methodology, techniques, and/or compositions employed herein.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measureable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

Recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “enhance,” “enhancing,” “improve” and “improving” (and grammatical variations thereof) describe an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 30%, 35%, 40%, 45%, 50%, 55%, 60%, 65%, 70%, 75%, 80%, 85%, 90%, 95%, 97%, 98%, 99%, or 100% such as compared to another measurable property or quantity (e.g., a control value). In some embodiments, the reduction can result in no or essentially no (i.e., an insignificant amount, e.g., less than about 10% or even 5%) detectable activity or amount.

As used herein “another” can mean at least a second or more.

The term “comprising”, which is synonymous with “including,” “containing,” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed subject matter can include the use of either of the other two terms.

All ranges disclosed herein are to be understood to encompass any and all subranges subsumed therein and any individual values therein. For example, a stated range of “1.0 to 10.0” should be considered to include any and all subranges beginning with a minimum value of 1.0 or more and ending with a maximum value of 10.0 or less, e.g., 1.0 to 5.3, or 4.7 to 10.0, or 3.6 to 7.9. Further, recitation of ranges of values herein are merely intended to serve as a shorthand method of referring individually to each separate value falling within the range, unless otherwise indicated herein, and each separate value is incorporated into the specification as if it were individually recited herein. For example, if the range 10 to 15 is disclosed, then 11, 12, 13, and 14 are also disclosed.

As used herein, phrases such as “between X and Y” and “between about X and Y” should be interpreted to include the values of X and Y. As used herein, phrases such as “between about X and Y” mean “between about X and about Y” and phrases such as “from about X to Y” mean “from about X to about Y.” All ranges disclosed herein are also to be considered to include the end points of the range, unless expressly stated otherwise. For example, a range of “between 5 and 10”, “from 5 to 10” or “5-10” should generally be considered to include the end points 5 and 10.

Further, when the phrase “up to” is used in connection with an amount or quantity, it is to be understood that the amount is at least a detectable amount or quantity. For example, a material present in an amount “up to” a specified amount can be present from a detectable amount and up to and including the specified amount.

As used herein, “oleogel” refers to a thermoreversible, semisolid composition and/or gel including a continuous liquid oil phase that is physically immobilized by a self-assembled network of oleogelators. An “oleogelator” as used herein refers to a compound that can form a self-assembled network in an oil. An oleogelator can impart specific qualities to an oil that the oil would not otherwise have in the absence of the oleogelator. In some embodiments, the presence of an oleogelator in an oil can form a thermoreversible three-dimensional gel or semisolid network to thereby provide an oleogel that traps liquid oil within the gel. The gel strength and/or rigidity of an oleogel may be a function of and/or dependent on the concentration of the oleogelator(s) present in the oleogel. Exemplary oleogelators include, but are not limited to, emulsifiers, small molecular weight molecules, and/or polymers. Further exemplary oleogelators include, but are not limited to, phospholipids or derivatives thereof, monoacylglycerols, diacylglycerols, lecithins (e.g., soy lecithin), pectins, fatty acids (e.g., a saturated fatty acid such as stearic acid), alginates (e.g., sodium alginate), and/or the like, and/or combinations thereof.

As used herein, an “emulsion” is a mixture of two or more immiscible phases (e.g., two or more immiscible liquids) where at least one phase contains a dispersion of another phase; generally, one phase comprises droplets (microscopic or ultramicroscopic in size) distributed throughout a second phase. In some embodiments, an oleogel can be one of the phases of an emulsion. An “oleogel emulsion” as used herein refers to an emulsion including an oleogel and a liquid phase that is immiscible with the oleogel.

As used herein, the phrase “bacterial cell mass” or “microbial cell mass” refers to an amount of bacterial cells or microbial cells, respectively, optionally an amount of probiotic cells. The amount of cells in a bacterial cell mass may be quantified, for example, at 9 Log CFU/mL.

As used herein, “probiotic” refers to a live microorganism (e.g., live bacterial cells) that can promote and/or provide one or more beneficial effect(s) and/or health benefit(s) when consumed by a subject (e.g., a mammal such as a human). In some embodiments, the beneficial effect(s) and/or health benefit(s) are increased when probiotics are consumed at high cell numbers, such as more than 6 Log CFU/mL, more than 7 Log CFU/mL, more than 8 Log CFU/mL, more than 9 Log CFU/mL, or more than 10 Log CFU/mL. “Microbial cells” generally refers to bacterial cells that are other than those defined as probiotic.

As used herein, “solid food” refers to a food or food product that is firm and stable in shape.

As used herein, “semi-solid food” refers to a food or food product that has a consistency between a solid and liquid. In some embodiments, a semi-solid food contains soft particles. Examples of a semi-solid food include, but are not limited to, yogurt, butter spreads, certain cheeses, and mayonnaise.

As used herein, “food grade” refers to a component that is edible, typically by humans. In some embodiments, a food grade component (e.g., compound) has no harmful effect on human health or has a GRAS (generally recognized as safe) status. In some embodiments, a food grade component is deemed safe for human consumption, optionally by the U.S. Food and Drug Administration.

An “active ingredient” as used herein refers any ingredient that can provide a beneficial effect (e.g., a health and/or therapeutic effect) to a subject (e.g., a human). Exemplary active ingredients include, but are not limited to, cells (e.g., microbial cells), proteins (e.g., enzymes), probiotics, vitamins, micronutrients, and/or antimicrobial agents. In some embodiments, the active ingredient is probiotic. In some embodiments, the active ingredient is a live microbial cell, optionally a live microbial cell that is not classified as a probiotic. In some embodiments, the active ingredient is an enzyme such as, but not limited to, a lactase and/or glucosidase.

In some embodiments, the presently disclosed subject matter provides a composition comprising: an active ingredient and an oleogel that comprises an oil, a wax, and an oleogelator, wherein the active ingredient is within the oleogel. The active ingredient may be encapsulated within the oleogel. In some embodiments, the active ingredient is selected from the group consisting of a probiotic, microbial cell, enzyme, vitamin, micronutrient, antimicrobial agent, and any combination thereof. In some embodiments, the probiotic comprises L. bulgaricus, L. reuteri, and/or L. rhamanos. In some embodiments, the enzyme is lactase, glucosidase, or combinations thereof. In some embodiments, the active ingredient is in an aqueous solution within the oleogel and/or composition. In some embodiments, the oil is selected from the group consisting of a sunflower oil, peanut oil, corn oil, canola oil, sesame oil, vegetable oil, hazelnut oil, olive oil, pomegranate seed oil, rapeseed oil, palm oil, soybean oil, coconut oil, avocado oil, walnut oil, flaxseed oil, safflower oil, almond oil, fish oil, algal oil, palm stearin, palm olein, palm kernel oil, butter oil, cocoa butter, cottonseed oil, mineral oil, and any combination thereof. In some embodiments, the oil is selected from the group comprising sunflower oil, peanut oil, corn oil, canola oil, and mixtures thereof. In some embodiments, the wax is selected from the group consisting of a rice bran wax, beeswax, carnauba wax, and any combination thereof In some embodiments, the oleogelator is selected from the group consisting of a phospholipid, phospholipid derivative, pectin, sodium alginate, lecithin, and any combination thereof. In some embodiments, the oleogelator is selected from the group consisting of a pectin, sodium alginate, lecithin, and any combination thereof. In some embodiments, the oleogelator is a phospholipid or a phospholipid derivative. In some embodiments, the composition further comprises lecithin, lactic acid, potassium citrate, and/or a protein. In some embodiments, the lecithin, lactic acid, potassium citrate, and/or protein is encapsulated with the active ingredient in an aqueous solution within the composition and/or oleogel. In some embodiments, the protein source is selected from the group consisting of buttermilk solids, milk, and soy, and amino acids from milk, soy or buttermilk. Without being bound by theory, the addition of lecithin or other oleogelator to a wax and oil mixture and of lecithin or other oleogelator to an aqueous solution including an active ingredient, such as a probiotic, may increase the shelf life of the active ingredient and/or the shelf life of the solid oleogel.

In some embodiments, the wax is present in the oleogel in an amount of about 1% to about 10% by weight of the oil present in the oleogel. In some embodiments, the composition is an oleogel emulsion.

In some embodiments, the composition further comprises an aqueous phase. In some embodiments, the composition comprises a total volume of an oil and wax together and a volume of an aqueous phase in a volume ratio of about 0.25:1 to about 6:1 (total volume of the oil and wax:volume of the aqueous phase), optionally wherein the volume ratio is about 1:4 or about 4:1 (total volume of the oil and wax:volume of the aqueous phase). In some embodiments, the composition comprises an aqueous phase and the oleogelator is present in an amount of about 0.1% to about 10% by volume of the aqueous phase, optionally wherein the oleogelator is present in the aqueous phase.

In some embodiments, the active ingredient is a probiotic and the composition comprises the probiotic in an amount of at least about 6 Log CFU/mL, at least about 7 Log CFU/mL, at least about 8 Log CFU/mL, at least about 9 Log CFU/mL, or at least about 10 Log CFU/mL. In some embodiments, at about 3 months after formation of the composition, the viability of the probiotic in the composition is at least about 6, 7, 8, 9, or 10 Log CFU/mL (i.e., the amount of surviving viable probiotics at about 3 months after formation of the composition is at least about 6, 7, 8, 9, or 10 Log CFU/mL. In some embodiments, at about 6 months after formation of the composition, the viability of the probiotic in the composition is at least about 6, 7, 8, 9, or 10 Log CFU/mL. (i.e., the amount of surviving viable probiotics at about 6 months after formation of the composition is at least about 6, 7, 8, 9, or 10 Log CFU/mL).

In some embodiments, the oleogel has a melting temperature of about 35° C. to about 50° C. In some embodiments, the composition is a food product.

In some embodiments, the presently disclosed subject matter provides a method of preparing a composition comprising an active ingredient, the method comprising: forming an oleogel comprising a wax, an oil, and an oleogelator; and combining an active ingredient and the oleogel to form the composition comprising the active ingredient.

In some embodiments, forming the oleogel comprises combining the wax and the oil to form a first mixture, heating the first mixture to form a liquid, and combining the liquid with an aqueous phase comprising the oleogelator. In some embodiments, the active ingredient is selected from the group consisting of a probiotic, microbial cell, enzyme, vitamin, micronutrient, antimicrobial agent, and any combination thereof. In some embodiments, the active ingredient is a probiotic. In some embodiments, the probiotic comprises L. bulgaricus, L. reuteri, and/or L. rhamanos. In some embodiments, the active ingredient is an enzyme such as a lactase, glucosidase or combinations thereof. In some embodiments, the active ingredient is in an aqueous solution within the oleogel and/or composition.

In some embodiments, the method further comprises, prior to combining the active ingredient and the oleogel, contacting the active ingredient to lecithin, lactic acid, potassium citrate, and/or a protein. In some embodiments, the composition further comprises lecithin, lactic acid, potassium citrate, and/or the protein. In some embodiments, the contact of the active ingredient and the lecithin, lactic acid, potassium citrate, and/or a protein is before the active ingredient is combined with (e.g., encapsulated in) the oleogel. In some embodiments, the lecithin, lactic acid, potassium citrate, and/or protein is/are encapsulated with the active ingredient in an aqueous solution within the oleogel and/or composition.

In some embodiments, the method further comprises combining the composition and a food product.

In some embodiments, the presently disclosed subject matter provides a method for the encapsulation of a bacterial cell mass, optionally a probiotic cell mass, in an oleogel comprising: (a) combining a wax, an oil and an oleogelator to form a mixture; (b) adding a bacterial cell mass, optionally a probiotic cell mass, comprising at least about 6 Log CFU/mL to the mixture; and (c) allowing the resulting mixture to solidify. In some embodiments, the combination of the wax, oil and oleogelator is heated until the mixture is fully liquid. In some embodiments, the bacterial cell mass comprises L. bulgaricus, L. reuteri, and/or L. rhamanos. In some embodiments, the oil is selected from the group comprising sunflower oil, peanut oil, corn oil, canola oil, and mixtures thereof. In some embodiments, the wax is selected from the group comprising rice bran wax, beeswax, carnauba wax, and mixtures thereof. In some embodiments, the oleogelator is selected from the group comprising pectin, sodium alginate, soy lecithin, and mixtures thereof. In some embodiments, the bacterial cell mass comprises at least about 7 Log CFU/mL, at least about 8 Log CFU/mL or at least about 9 Log CFU/mL.

In some embodiments, the presently disclosed subject matter provides a composition comprising a bacterial cell mass, optionally probiotic cells, encapsulated in an oleogel, wherein said oleogel comprises a wax, an oil and an oleogelator, wherein the cell mass comprises at least about 6 Log CFU/mL. In some embodiments, the oil is selected from the group comprising sunflower oil, peanut oil, corn oil, canola oil, and mixtures thereof. In some embodiments, the wax is selected from the group comprising rice bran wax, beeswax, carnauba wax, and mixtures thereof. In some embodiments, the oleogelator is selected from the group comprising pectin, sodium alginate, soy lecithin and mixtures thereof. In some embodiments, the oleogel comprises beeswax, corn oil, and pectin. In some embodiments, the bacterial cell mass comprises at least about 7 Log CFU/mL, at least about 8 Log CFU/mL or at least about 9 Log CFU/mL. In some variations, the composition is at least about 3 months old, at least about 6 months old, at least about 12 months old, at least about 18 months old or at least about 24 months old. In some embodiments, the composition has been stored at room temperature, generally about 20° C., or at about 4° C., or between about 4° C. and about 25° C. In some embodiments, the bacterial cell mass comprises L. bulgaricus, L. reuteri, and/or L. rhamanos.

In some embodiments, a composition of the present invention is a food product such as, but not limited to, a solid or semi-solid food product. In some embodiments, the food product is a dairy product, such as for example, yogurt or cheese. In some embodiments, the food product is a plant-based food product or an animal-based food product.

Oleogelation involves restructuring liquid oil into a semisolid fat. The additives that restructure the oil, known as oleogelators, impart specific qualities that an oil would not otherwise have, and can be used to form a thermoreversible three-dimensional gel network, called an oleogel, that traps liquid oil within a gel. Oleogels are typically semisolid and the strength of the gel is usually a function of the concentration of the organogelator(s). Oleogels have a fatty acid profile that resembles their base oils. The semi-solid nature of the gel results in physiochemical attributes closer to a saturated or trans-fat product.

Vegetable oils are examples of organic materials that can be structured into oleogels using relatively low concentrations (<10%) of oleogelator. Oleogelators can gel unsaturated oils at both refrigeration and ambient temperatures, enabling them to be effective contributions in processed foods. In such uses as disclosed herein, oleogel components are typically composed of agents commonly used in food products. For example, peanut oil, sunflower oil, and corn oil, can each be used due to their stability, chemical composition, economic importance, availability, and cost. One example of oleogelation is the addition of an oleogelelator mixture of soy lecithin or stearic acid and water to a base of canola oil and a wax, converting the oil into an oleogel.

Examples of food grade oil that can be a component of an oleogel include but are not limited to, soybean oil, canola oil, corn oil, sunflower oil, safflower oil, flaxseed oil, almond oil, peanut oil, fish oil, algal oil, palm oil, palm stearin, palm olein, palm kernel oil, butter oil, cocoa butter, avocado oil, almond oil, coconut oil, cottonseed oil, and/or mineral oil.

In some embodiments, bacterial cells are grown in MRS broth before being isolated and transferred to an oleogel. Other methods to grow bacteria and growth media are well-known to those of skill in the art and may be used. In some embodiments, all of the cells isolated from the MRS broth are used in preparation of the oleogel. In some embodiments, cells are selected based on size, such using a 10,000 Dalton cutoff and isolating above this cutoff. Cells can be isolated using ultrafiltration or other methods known to those of skill in the art, such as centrifugation.

In some embodiments of the present application, isolated cells can be exposed to a lecithin, amino acid, and/or protein. As generally disclosed herein, lecithin can be isolated from soybeans (soy lecithin) or from a food such as egg yolk, fish, avocado, sunflower seeds, meat such as liver, dairy such as milk or buttermilk solids. Exemplary protein sources include, but are not limited to, buttermilk solids, milk, and soy, and exemplary amino acids include, but are not limited to, those from milk, soy, and/or buttermilk. Buttermilk solids, containing both lecithin and protein, can conveniently be reconstituted in solution, but other sources can be used according to the methods disclosed herein. Without being bound by theory, exposure to a lecithin, amino acid, and/or protein in an acidic solution (e.g., pH about 5.5 to about 6) may improve the stability of the bacterial cells, thereby increasing the shelf life of the bacteria once incorporated into the oleogel formulation.

As shown herein, it is possible to store viable probiotic cells in a solidified oleogel comprised of a wax, an oil, an oleogelator, and water. The oleogel can be incorporated into a solid or semi-solid food product, such as yogurt or cheese according to methods known to those of skill in the art and consistent with the production of such food products.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Determination of Water Activity

Water activity of samples was measured using Aqualab 4 TE water activity meter (Meter Group, Inc., Pullman, Wash.) at 25° C.

### Example 1

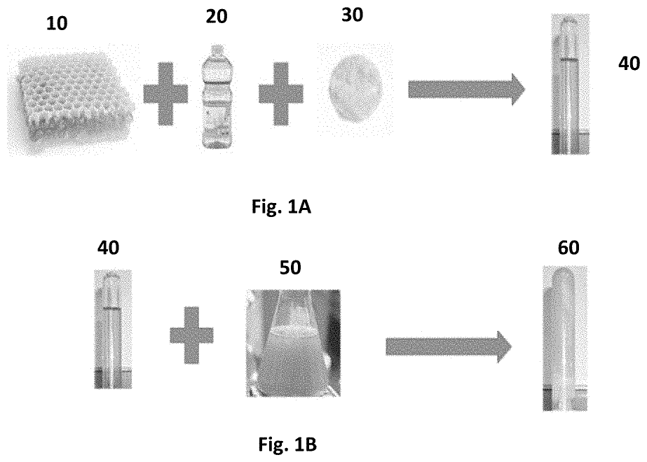

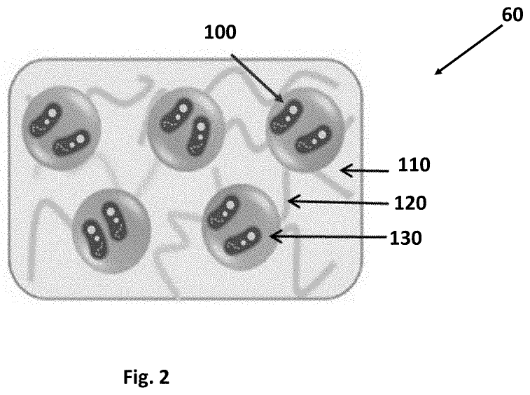

As disclosed herein, and as generally shown in FIG. 1A, a wax 10, an oil 20, and an oleogelator 30, e.g. pectin, were combined and the mixture heated in a waterbath until the mixture was liquid 40. As generally shown in FIG. 1B, the liquid mixture 40 was then mixed with a bacterial culture 50 comprising a probiotic (e.g., L. bulgaricus) and the entire composition was allowed to solidify at room temperature yielding the solid product 60. FIG. 2 illustrates an exemplary oleogel 60 comprising the bacteria 100 in an aqueous phase 130 encapsulated in a continuous oil phase 110, in the presence of 120, a water/oil emulsifier.

The oleogel formulation (100 mL scale preparation) was prepared in the following manner: 50 mL of 5% (w/w) of beeswax (Jedwards International, Braintree Mass.)/corn oil (Mazola, purchased from Harris Teeter) mixtures were prepared, heated to 48° C. until the mix was liquefied and then mixed at a 1:4 ratio (v/v) of a 5% (w/v) solution of pectin (Lille Skensved) dissolved in water.

Fifty mL of L. bulgaricus S9 (Food Biotechnology Lab, North Carolina Agricultural & Technical State University, Greensboro, N.C.) was cultivated at 37° C. in MRS Broth (NEOGEN Culture Media, Neogen, Lansing, Mich.) for 16 hours to at least about 9 log CFU/mL. The culture was then centrifuged at 4500 g at 4° C. and the precipitate resuspended in 2 mL peptone water, prepared by dissolving 1 g Bactoprotease Peptone #3 (BD Biosciences, San Jose, Calif.) in 1 L of deionized water and autoclaving the mixture at 121° C. for 15 minutes. The L. bulgaricus+peptone water suspension was mixed at 48° C. with the beeswax/oil/pectin oleogel. Droplets of the bacteria/beeswax/oil/pectin mixture were then pipetted into a 5% solution of CaCl₂ in deionized in water. The droplets all solidified into generally oval shapes, which were collected and held at −20° C. for 10 minutes and then stored in a closed container at 22° C. unless otherwise noted.

On day 1 and day 8, the viability of the cells stored in the oleogel was determined according to the following method: the oleogel/bacteria mixture (20 g) was heated to 48° C. and diluted with 1 mL Tween 20 (Thermo Fisher Scientific, Waltham, Mass.). The mixture was centrifuged, then diluted with 90 mL MRS broth that had been preheated to 48° C. The MRS-oleogel mixture was then serially diluted six times with peptone water. Each serial dilution step consisted of a 1:9 (v/v) of the previous dilution into peptone (10 mL total volume). The last of the serial dilutions was then spread on MRS-Agar plates (prepared by combining 55 g MRS powder (Accumedia), 0.5 g cysteine (Thermo Fisher Scientific, Waltham, Mass.), 16 g technical agar (Thermo Fisher Scientific, Waltham, Mass.), with 1 L of deionized water. This mixture was autoclaved at 121° C. for fifteen minutes, cooled to 45° C., aliquoted into petri dishes, cooled to room temperature and stored at 4° C. until use.) The treated plates were incubated at 42° C. for 24 hours. The resulting colonies on the plates were counted (Log Colony Forming Units per mL, or Log CFU/mL) to determine the number of viable cells in the oleogel/bacteria mixture.

According to the methods disclosed herein, at least about 9 Log CFU/mL of L. bulgaricus S9 were successfully incorporated into an oleogel in the solid phase, based on an analysis of data from a rheometer (Discovery HR20, TA Instruments, New Castle, Del.). On each of the first seven days, 9 Log CFU/mL were detected according to the method disclosed above. Upon plating the oleogel on MRS/agar on the 8^{th} day, 8 Log CFU/mL of L. bulgaricus S9 were recovered, demonstrating the promising probiotic carrying capacity of the oleogel.

### Example 2

Three strains of Lactobacillus bulgaricus (S9, LB 6 and S23, each from the North Carolina Agricultural & Technical State University culture collection) were incorporated into an oleogel system comprising beeswax, corn oil, pectin and water, according to the method of Example 1.

The bacterial populations were stored at 4° C. or at 20° C. and were monitored every day for the first 7 days and then every 14 days for the next 140 days. The initial bacterial populations were between 9.0 and 10.2 Log CFU/ml. Until the end of testing at day 182, the bacterial counts were above 8 Log CFU/ml. Water activity remained within 0.2 to 0.3.

### Example 3

Lactobacillus bulgaricus LB 6 was incorporated into a 10 L oleogel formulation comprising beeswax, corn oil, pectin and water, following the method described in Example 1.

The initial count of bacterial population was 11 Log CFU/ml. The oleogel sample was kept at 4° C. for 6 months and after storage the bacterial population was determined to be between 9.2 and 9.5 Log CFU/ml.

### Example 4

Fifty mL L. bulgaricus LB 6 was cultivated at 37° C. in MRS Broth (NEOGEN Culture Media, Neogen, Lansing, Mich.) for 16 hours to 10.6 Log CFU/mL. Using a 10,000 Da, cutoff, the cells were collected using ultrafiltration. The cell were then washed with a 5.8 pH solution comprising lactic acid, potassium citrate (2%) and buttermilk solids (6-7% buttermilk solids powder reconstituted in solution), thereby exposing the cells to lecithin and protein.

The bacterial cell solution, containing lecithin and potassium citrate was incorporated into the oleogel as described in Example 1, comprising beeswax, corn oil, pectin and water.

The sample was kept at 4° C. for 6 months and upon testing the bacterial population was at least about 9.5 Log CFU/ml.

### Example 5

The method of preparation of an oleogel as described in Example 1, was followed, except sodium alginate (1% solution Thermo Fisher Scientific, Waltham, Mass.) was used in place of pectin.

Lactobacillus bulgaricus LB 6 was incorporated into the sodium alginate-containing oleogel. The initial cell count was 10 Log CFU/mL. After storage for 15 weeks at either 4° C. or 20° C., the cell count was measured and found to be about 8 Log CFU/mL. Water activity remained low (A_{w} 0.25) and upon visual observation little separation of the oil was observed and deemed too little to measure.

### Example 6

The method of preparation of an oleogel as described in Example 1, was followed, except extra virgin olive oil (Hacendado, Sevilla Spain) was used in place of corn oil.

Lactobacillus bulgaricus LB 6 was incorporated into the olive-oil containing oleogel. The initial cell count was 10 Log CFU/mL. After storage for 2 weeks at 4° C., the cell count was measured and found to drop below 5 Log CFU/mL; no further testing was conducted.

### Example 7

The delivery technology disclosed herein is capable of carrying a large number bacterial cells (probiotics) isolated from their surrounding environment. As disclosed herein, the oleogels (1) are made of food grade components, (2) are structurally stable (e.g., a solid state that does not collapse or otherwise become fluid), (3) have a high probiotic carrying capacity, (4) support the viability of encapsulated probiotics over time; (5) have a melting temperature of 40-48° C. so that it can be safely combined with bacterial cultures, and (6) have a solid at room temperature, so that the material can be physically manipulated.

Different formulations of oleogels as prepared herein can encapsulate probiotics, including strains of L. bulgaricus, L. reuteri, L. rhamanos and other probiotic strains.

### Preparing the Probiotic Oleogel

The following oleogel structuring agents are combined:
1) Base oil: sunflower oil, peanut oil, corn oil, or canola oil;
2) Wax: rice bran wax, beeswax or carnauba wax; and
3) Oleogelator: pectin; sodium alginate; egg yolk; lecithin, including but not limited to soy lecithin; or another phospholipid.

To obtain an oleogel, the methods disclosed herein are generally followed: 100 g oil is heated to 100° C. in an oven, generally resulting in the destruction of their crystallinity. The wax is added to the oil to a final concentration 1%-10% (mass wax/mass oil); and then mixed at a 1:4 ratio (v/v) with a 5% (w/v) solution of oleogelator dissolved in water. The resulting oleogel, still in a liquid state, is transferred to test tubes held in water baths of 40-48° C. A bacterial cell culture, generally at least 9 Log CFU/mL is added to the oleogelator mixture at a final concentration of 1-25% (v/v) and the mixture is left to solidify at room temperature.

### Determining Probiotic Cell Viability

The number of viable cells stored in the oleogel formulations are tested at each of one month, 2 months, 3 months, 4 months, 5 months, 6 months, 7 months, 8 months, 9 months, 10 months, 11 months, and 12 months using the methods described herein.

It will be understood that various details of the presently disclosed subject matter may be changed without departing from the scope of the presently disclosed subject matter. Furthermore, the foregoing description is for the purpose of illustration only, and not for the purpose of limitation.

That which is claimed is:

1. A composition comprising:
an active ingredient; and
an oleogel comprising an oil, a wax, and an oleogelator,
wherein the active ingredient is within the oleogel.

2. The composition of claim 1, wherein the active ingredient is selected from the group consisting of a probiotic, microbial cell, enzyme, vitamin, micronutrient, antimicrobial agent, and any combination thereof.

3. The composition of claim 1, wherein the oil is selected from the group consisting of a sunflower oil, peanut oil, corn oil, canola oil, sesame oil, vegetable oil, hazelnut oil, olive oil, pomegranate seed oil, rapeseed oil, palm oil, soybean oil, coconut oil, avocado oil, walnut oil, flaxseed oil, safflower oil, almond oil, fish oil, algal oil, palm stearin, palm olein, palm kernel oil, butter oil, cocoa butter, cottonseed oil, mineral oil, and any combination thereof.

4. The composition of claim 1, wherein the wax is selected from the group consisting of a rice bran wax, beeswax, carnauba wax, and any combination thereof.

5. The composition of claim 1, wherein the oleogelator is selected from the group consisting of a pectin, sodium alginate, lecithin, and any combination thereof or derivatives thereof.

6. The composition of claim 1, wherein the wax is present in the oleogel in an amount of about 1% to about 10% by weight of the oil present in the oleogel.

7. The composition of claim 1, wherein the composition is an oleogel emulsion.

8. The composition of claim 1, further comprising an aqueous phase and wherein the composition comprises the total volume of the oil and wax together and the volume of the aqueous phase in a volume ratio of about 0.25:1 to about 6:1 (total volume of the oil and wax:volume of the aqueous phase).

9. The composition of claim 1, further comprising an aqueous phase and wherein the oleogelator is present in the aqueous phase in an amount of about 0.1% to about 10% by volume of the aqueous phase.

10. The composition of claim 1, wherein the active ingredient is a probiotic and the composition comprises the probiotic in an amount of at least about 6 Log CFU/mL.

11. The composition of claim 10, wherein, at about 3 months after formation of the composition, the viability of the probiotic in the composition is at least about 8 Log CFU/mL.

12. The composition of claim 10, wherein, at about 6 months after formation of the composition, the viability of the probiotic in the composition is at least about 8 Log CFU/mL.

13. The composition of claim 1, wherein the oleogel has a melting temperature of about 35° C. to about 50° C.

14. The composition of claim 1, wherein the composition is a food product.

15. A method of preparing a composition comprising an active ingredient, the method comprising:
forming an oleogel comprising a wax, an oil, and an oleogelator; and
combining an active ingredient and the oleogel to form the composition comprising the active ingredient.

16. The method of claim 15, wherein forming the oleogel comprising combining the wax and the oil to form a first mixture, heating the first mixture to form a liquid, and combining the liquid with an aqueous phase comprising the oleogelator.

17. The method of claim 15, wherein the active ingredient is a probiotic.

18. The method of claim 15, further comprising, prior to combining the active ingredient and the oleogel, contacting the active ingredient to lecithin, lactic acid, potassium citrate, and/or a protein.

19. The method of claim 18, wherein the composition further comprises lecithin, lactic acid, potassium citrate, and/or the protein.

20. The method of claim 15, further comprising combining the composition and a food product.
